(19) United States
(12) Patent Application Publication
(10) Pub. No.: US 2009/0180974 A1
(43) Pub. Date: Jul. 16, 2009

(54) Agent For Protecting Body Cells

(76) Inventor: Hans-Emanuel Holzgang, Zurich (CH)

Correspondence Address: FITCH EVEN TABIN & FLANNERY, 120 SOUTH LASALLE STREET, SUITE 1600, CHICAGO, IL, 60603-3406, US

(21) Appl. No.: 12/092,394

(22) PCT Filed: Oct. 23, 2006
(86) PCT No.: PCT/CH06/00591

§ 371 Date: Dec. 5, 2008

(30) Foreign Application Priority Data
Nov. 2, 2005 (CH) 1755/05

(51) Int. Cl. A61K 8/49; A61K 31/353; A61K 31/355; A61K 31/34; A61K 31/07; A61K 38/43; A61Q 17/04

(52) U.S. Cl. 424/59; 514/456; 514/458; 514/474; 514/725; 424/94.1

(57) ABSTRACT

The invention relates to an agent or a composition for protecting body cells for the preventive and/or performance-enhancing influencing of body cells. The agent comprises a combination of herbal active substances and vitamins and amino acids, with one active substance consisting of polyphenols from green tea.

The invention relates to an agent for protecting body cells, in particular for protecting skin cells.

DE-U-29604205 discloses a skin protection spray with high water content and plant extracts such as meristem extract or alpha-bisabolol, which serve as free-radical scavengers, and also vitamin E or vitamin derivatives. The high water fraction stems both from the plant extracts and also added water. The high-water-content solution is said to cool and avoid a sticky-greasy impression on the skin.

A skin care composition according to DE-U-20204160 comprises at least one active ingredient of an algae, active ingredients of Bupleurum Chinensis (root), and vitamins A, C and/or E. It is said to develop a prophylactic effect in the case of stress due to free radicals and a restructuring effect in the case of photoaging. Dermatological findings, such as itching or flaking, are likewise said to be reduced.

A further natural-based skin care agent is described in DE-A-4318280 which comprises extracts of marigold flowers, chamomile flowers and optionally further plants such as, for example, elm bark or linseed. The composition is said to serve the cosmetic treatment of skin irregularities, including psoriasis or neuro-dermatitis.

The object of the invention is to develop a further agent for protecting body cells which can develop both a preventive and also a performance-enhancing effect. The object is achieved with the features of patent claim 1.

The agent or the composition comprises combined green tea extracts, in particular green tea polyphenols, which rapidly soak into the skin and can likewise be readily absorbed. These are extracted and stabilized in such a way that, even when applied in a very low dose, they exhibit a protective effect.

Preferably, the combination also comprises the vitamins A, C and/or E and/or vegetable isoflavones, for example red clover, in order to achieve a synergistic effect.

Possible applications arise in combination with:
UV filters for sunscreen compositions, as a result of which the effectiveness of the protection in the case of UV-induced skin damage is increased. A combination with chemical or physical filters (e.g. titanium dioxide, zinc oxide) is possible, the concentration of the agent in these should be at least 0.4% in the end product (should the sun filter constitute, for example, 5% of a sunscreen lotion or the like, about 5-10% of the filter must comprise the agent according to the invention). Individual filters can also be combined with one another and with the agent;
further substances, such as, for example, vitamins, coenzymes, vitamin derivatives, trace elements, enzymes, amino acids or substances which stimulate the endogenous production of such.

Further possible uses are as:
cortisone replacement (replacement of cortisone or cortisone-like synthetic substances for application to the skin or by inhalation and use also to combat inflammatory diseases such as arthritis or inflammation of the veins,
application for increasing mental and physical performance, in connection with Alzheimer's therapies, chelating agents or in combination with DMSA etc.
use in skin protection products, skin protection during radiation therapies, protection of personnel, protection against cell damage during radioactive or electromagnetic radiation;
use in the case of ulcers, diabetes patients or wherever corticoids are used.

Accordingly, the agent can be very readily used as skin or cell protection and as free-radical scavenger also in foods (likewise for enhancing performance), i.e. is suitable for external and internal therapeutic and medicinal use.

Also conceivable is a replacement of tannins for alleviating itching in the case of infections (e.g. chickenpox or rubella), where other substances, such as, for example, zinc oxide are also applied.

On account of the antiviral activity, application in the case of grippal infections (influenza, HIV, herpes, bird flu or the like) likewise appears possible, also in combinations which support the antiviral activity. Further applications are in cancer therapy in combination with taxol, AC, CMF or EC therapies or mistletoe therapy.

In order to improve the effect of the agent, derivatives of these can be formed, including protection of functional groups or individual molecules, their substitution or the addition of other compounds. Conversely, individual constituents may also be synthesized or condensed to give a new molecule in order to combine this in turn with the agent and to optimize its overall effect.

In order to avoid possible side-effects, the agent is free from organic solvents and comprises preferably only water as solvent.

The agent for oral or topical use can advantageously be microencapsulated or nanoencapsulated.

The invention also provides, according to claim 9, a method of producing the agent.

Preferably, the extraction using water takes place at temperatures in the range from 50° C. to 120° C. and at pressures in the range from 1 bar to 5 bar. A cost-effective variant operates at ambient pressure, about 1 bar absolute, and uses temperatures in the range from 50° C. to 98° C. If required, it is also possible to work at superatmospheric pressure, between 1 bar absolute and 5 bar absolute, in which case temperatures between 50° C. and 120° C. are used.

Expediently, the filtration takes place by means of a filter bed, where the filter bed used is an oxide bed filter, a polymer bed filter or mixed forms of these two filter types. Particular preference is given to porous or particulate silica gel filters, glass fiber filters, cellulose filters or inorganic-organic mixed forms, such as silica gel with grafted-on organic groups, such as, for example, alkyl groups.

In a particularly preferred embodiment, the filter bed comprises at least one stationary filter bed phase made of nonpolar material, in particular a porous silica gel structure with silica gel particles whose surfaces are equipped with nonpolar organic groups (so-called reversed phase silica gels). As a result, during filtration, the lipophilic fractions of the extract are removed, giving a green tea extract which is free from organic solvents.

The pore sizes and/or particle sizes or fiber diameters of the filter bed used are expediently in the range from 0.01 mm to 0.5 mm and preferably in the range from 0.05 mm to 0.2 mm. Particular preference is also given to polydisperse filter bed packings, in particular with nonpolar surfaces.

Via the adjustment and selection of the abovementioned filter parameters, such as, for example, particle sizes, pore sizes, polar or nonpolar filter material, it is possible to adjust the spectrum of the amino acid fractions of the agent according to the invention.

During subsequent concentration of the extract, a concentration of ingredients other than water to 40% by weight to 100% by weight takes place. As a result of moderate evaporation, the extract is thickened to give a thin-liquid to thick-liquid syrup with 40% by weight to 80% by weight of ingredients (substances other than water). Alternatively, spray-drying or drum-drying of the extract can be carried out, in which case enrichment is to 90% by weight to 100% by weight of ingredients in the abovementioned sense. The abovementioned percentages by weight (% by wt.) are to be understood as meaning the mass of the sum of all of the ingredients other than water, based on the total mass.

Surprisingly, it has also become evident that the preparation method according to the invention leads to an agent whose glutamate fraction or glutamic acid fraction of the overall amino acid fraction is greater than 60%, in particular between 60% and 90%.

In the case of a particularly effective embodiment (“amino acid spectrum”) of the agent according to the invention, the relative fractions of certain amino acids (i.e. the weight fractions of the particular amino agent based on the total mass of all of the amino acids present in the agent) are in the following ranges:

| | |
|---|---|
| Glutamic acid: | 60%-80% |
| Aspartic acid: | 10%-14% |

and in particular:

| | |
|---|---|
| Serine: | 2%-4% |
| Glycine: | 2%-4% |
| Alanine: | 2%-3% |
| Valine: | 1%-3% |
| Threonine: | 1%-3% |

A further object of the invention consists in indicating a therapeutic method for protecting and for influencing body cells and for the use of an agent for this purpose.

The agent can both be used dermatologically and also be taken orally. The dermatological use can be cosmetic in nature (antiaging) or medicinal in nature (suppression of inflammations, cancer prevention, influencing of the immune system on the skin or influencing of endogenous repair mechanisms). The taking of the agent can likewise be medicinal in nature with the abovementioned fields of use, plus allergy reduction. Furthermore, the agent can be taken as food supplement for the purpose of increasing performance, antiaging, weight reduction or generally for protecting health.

Application of the agent is possible with the customary administration forms, for example in the form of a cream, spray, capsule or drink.

A further use aspect of the agent is its use as gene modulator for enzymes, for example apoptosis promoters are influenced at gene level (e.g. regulation of matrix metalloproteinases MMP1 or MMP2).

The invention is described in more detail below in a working example. A white, stabilized skin cream comprises green tea extracts with stabilized reactive groups (polyphenols) and the vitamins A, C and E for regular application to areas of skin, for example with age spots or melanomas. The active ingredients are contained only in aqueous solution.

Even after application for a short time, a retrogression of damaged cells or an isolation of such cells or a freezing of the existing level is evident. An antioxidative effect can be detected and a dermatological protection or a protection of genetic information in affected body cells or skin cells can clearly be achieved.

1-20. (canceled)

21. A therapeutic agent comprising green tea extract and vitamins, wherein the green tea extract includes green tea polyphenols and an amino acid fraction, wherein the amino acid fraction includes a glutamate fraction selected from the group consisting of glutamate and glutamic acid, wherein the glutamate fraction is 60 to 90 percent by weight of the amino acid fraction.

22. The agent as claimed in claim 21, wherein the vitamins are selected from the group consisting of vitamin A, vitamin C, vitamin E, and mixtures thereof.

23. The agent as claimed in claim 21, wherein the agent further comprises chemical or physical UV-filters.

24. The agent as claimed in claim 21, wherein the agent further comprises at least one compound selected from the group consisting of isoflavone, flavonoid, catechin, proanthocyanidine, enzyme, coenzyme, vitamin derivative, amino acid, and trace element.

25. A method for protecting and therapeutically treating the skin, the method comprising topically applying a therapeutic agent comprising green tea extract and vitamins, wherein the green tea extract includes green tea polyphenols and an amino acid fraction, wherein the amino acid fraction includes a glutamate fraction selected from the group consisting of glutamate and glutamic acid, wherein the glutamate fraction is 60 to 90 percent by weight of the amino acid fraction.

26. The method of claim 25, wherein the vitamins are selected from the group consisting of vitamin A, vitamin C, vitamin E, and mixtures thereof.

27. The method of claim 25, wherein the agent further comprises chemical or physical UV-filters.

28. The method of claim 25, wherein the agent further comprises at least one compound selected from the group consisting of isoflavone, flavonoid, catechin, proanthocyanidine, enzyme, coenzyme, vitamin derivative, amino acid, and trace element.

29. A method for reducing the amount of cortisone used in a therapeutic agent, the method comprising substituting a therapeutic agent for the cortisone, the therapeutic agent comprising green tea extract and vitamins, wherein the green tea extract includes green tea polyphenols and an amino acid fraction, wherein the amino acid fraction includes a glutamate fraction selected from the group consisting of glutamate and glutamic acid, wherein the glutamate fraction is 60 to 90 percent by weight of the amino acid fraction.

30. The method of claim 29, wherein the vitamins are selected from the group consisting of vitamin A, vitamin C, vitamin E, and mixtures thereof.

31. The method of claim 29, wherein the agent further comprises at least one compound selected from the group consisting of isoflavone, flavonoid, catechin, proanthocyanidine, enzyme, coenzyme, vitamin derivative, amino acid, and trace element.

32. A method for protecting the skin during exposure of the skin to ultraviolet rays, the method comprising applying a therapeutic agent to the skin, the therapeutic agent comprising green tea extract and vitamins, wherein the green tea extract includes green tea polyphenols and an amino acid fraction, wherein the amino acid fraction includes a glutamate fraction selected from the group consisting of glutamate and glutamic acid, wherein the glutamate fraction is 60 to 90 percent by weight of the amino acid fraction.

33. The method of claim 32, wherein the vitamins are selected from the group consisting of vitamin A, vitamin C, vitamin E, and mixtures thereof.

34. The method of claim 32, wherein the agent further comprises chemical or physical UV-filters.

35. The method of claim 32, wherein the agent further comprises at least one compound selected from the group consisting of isoflavone, flavonoid, catechin, proanthocyanidine, enzyme, coenzyme, vitamin derivative, amino acid, and trace element.

36. The method of claim 32, wherein the glutamate fraction is 60 to 80 percent by weight of the amino acid fraction

37. A method for making a therapeutic agent, the method comprising:
extracting green tea polyphenols and vitamins from green tea leaves using water;
filtering the extract; and
concentrating the extract.

38. The method of claim 37, wherein the extraction step takes place at a temperature in the range from 50° C. to 120° C. and a pressure in the range from 1 bar to 5 bar.

39. The method of claim 37, further comprising stabilizing the green tea polyphenols.

40. The method of claim 37, wherein a filter bed is used for the filtration step, the filter bed selected from the group consisting of an oxide bed filter and a polymer bed filter.
